(19) United States
(12) Patent Application Publication
(10) Pub. No.: US 2014/0371868 A1
(43) Pub. Date: Dec. 18, 2014

(54) Modular Joint Prosthesis System, Premounted Joint Prosthesis System Part and Sterile Article

(71) Applicant: Merete Medical GmbH, Berlin (DE)

(72) Inventor: Emmanuel Anapliotis, Berlin (DE)

(73) Assignee: Merete Medical GmbH, Berlin (DE)

(21) Appl. No.: 14/316,045

(22) Filed: Jun. 26, 2014

Related U.S. Application Data
(62) Division of application No. 13/817,407, filed on Feb. 15, 2013.

(30) Foreign Application Priority Data
Oct. 18, 2010 (DE) 202010008828.3

(51) Int. Cl. A61F 2/32; A61F 2/36

(52) U.S. Cl. CPC A61F 2/32; A61F 2/3609; A61F 2002/30344
USPC 623/23.12

(57) ABSTRACT

A modular hip joint prosthesis system has an intermediate element which has an inner surface for receiving a prosthesis neck and an outer surface, a joint ball head which is embodied as a plug head having an inner surface associated with the outer surface of the intermediate element, and a bipolar joint socket in which an inlay is received in an outer shell in a freely moving manner and which has a ball receiver which is associated with the joint ball head, configured such that the joint ball head can be received in a movable manner. A premounted joint prosthesis system part for a modular joint prosthesis system and a sterile article are also provided.

### REFERENCE TO RELATED APPLICATION

The present application is a divisional of U.S. patent application Ser. No. 13/817,407, filed Feb. 15, 2012, which application is the US. national stage application of International Application No. PCT/DE2011/075250, filed Oct. 12, 2011. The international application claims priority of German Patent Application No. 20 2010 008 828.3, Filed Oct. 18, 2010. All of the above mentioned applications and patents are incorporated herein by reference.

The invention relates to a modular joint prosthesis system, in particular a modular hip joint prosthesis system, a pre-assembled joint prosthesis system part and a sterile article.

### BACKGROUND OF THE INVENTION

Joint prosthesis systems are known in various embodiments. Modular systems usually have a joint socket which is formed with a ball receptacle for a joint ball head. In use, the joint ball head itself is connected to a prosthesis neck, which may be effected directly or with the aid of intermediate elements. In this way a rigid connection is produced between the prosthesis neck and the joint ball head. The joint ball head itself is freely movably received in the joint socket.

The document DE 91 03 574 U1 discloses a modular joint prosthesis system which consists of an intermediate element, a joint ball and a prosthesis shaft with a neck to receive the joint ball. The intermediate element can be inserted between the prosthesis shaft and the joint ball. The external contour of the intermediate element is adapted to the internal contour of a blind bore in the joint ball. The internal contour of the intermediate element corresponds to the external contour of the prosthesis neck. The internal contour and the external contour of the intermediate element are not formed axially with respect to one another.

Furthermore, a modular joint prosthesis system is known from the document WO 2005/089676 A1, wherein a plurality of intermediate elements which are inserted into one another are disposed between the prosthesis shaft and the joint ball.

Modular joint prosthesis systems of the aforesaid type are for example marketed under the trade name BioBall. They make it possible to offset the center point of the joint ball head relative to the longitudinal axis of the prosthesis neck. In this way an individual adaptation to the patient is possible.

In connection with hip endoprosthesis operations, post-operative dislocations frequently cause problems which can lead to significant complications. Therefore there is a need for improved joint prosthesis systems.

### BRIEF SUMMARY OF THE INVENTION

The object of the invention is to provide improved technologies for a modular joint prosthesis system, in particular a modular hip joint prosthesis system, which allow the prosthesis system to be adapted individually to the patient facilitate and moreover reduce the probability of dislocations. Furthermore the probability of complications occurring during author after implantation of the joint prosthesis in the patient's body are reduced.

This object is achieved by a modular joint prosthesis system, in particular a modular hip joint prosthesis system. Furthermore in order to achieve this object a pre-assembled joint prosthesis system part and a sterile article are provided. Advantageous embodiments are disclosed.

The invention encompasses the idea of a modular joint prosthesis system, in particular a modular hip joint prosthesis system, with:
an intermediate element which has an inner cone or surface to receive a prosthesis neck and an outer cone or surface,
a joint ball head which is formed as a plug head with an internal cone associated with the outer cone or surface of the intermediate element, and
a bipolar joint socket, in which an inlay is freely movably received in an outer shell and which has a ball receptacle associated with the joint ball head and configured to movably receive the joint ball head.

According to a further feature, a pre-assembled joint prosthesis system part for a modular joint prosthesis system is provided, in particular a joint prosthesis system of the aforesaid type, with the following features:
an inlay made of a plastic material which is associated with a bipolar joint socket, is formed with a ball receptacle formed and is configured so as to be freely movably received in an outer shell of the bipolar joint socket, and
a joint ball head which is movably disposed as a plug head in the ball receptacle of the inlay and is formed with an inner cone or surface.

Finally, the invention encompasses the idea of a sterile article with a pre-assembled joint prosthesis system part which is received in a sterile pack.

The proposed joint prosthesis system combines both the individual adaptability of the prosthesis system to different patients, which is facilitated in particular with the aid of the intermediate element and the specific characteristics thereof, and also the advantage of a reduced probability of dislocation, in that the joint socket which receives the joint ball head is bipolar. The bipolar configuration means in particular that an inlay which itself movably receives the joint ball head is freely movably received in an outer shell. To this end the joint ball head snaps into the associated ball receptacle. The installation of the joint ball head is performed preferably by means of a manual pressing tool by which the joint ball head is pressed into the ball receptacle. In this way on the one hand a firm connection is provided between the joint ball head and the receptacle and on the other hand a type of dual mobility is provided. The mobility of the inlay within the outer shell leads to a clear reduction in the shear forces on the contact surface between the bone and the outer shell and thus has a favorable effect on the long-term stability of the implant. The known advantages associated with the use of the intermediate element per se, in particular the individual adaptability to the patient and the resulting reduction in post-operative problems, are optimally developed on the basis of the use of the bipolar joint socket.

With the aid of the pre-assembled joint prosthesis system part a module for the joint prosthesis is provided, the use of which makes it possible during implantation for the doctor in particular to economize on the step of pressing in the joint ball head into the ball receptacle of the inlay in the operating field, which is regularly carried out with a manual pressing tool. The pre-assembled joint prosthesis system part can be provided in a sterile pack. Contaminations, which can occur in known systems during implantation in situ when the joint ball head is pressed in, are ruled out.

In an advantageous embodiment the invention provides that the inner cone and the outer cone of the intermediate element are not formed coaxially with respect to one another.

A preferred modification provides that the intermediate element is of multi-part construction, wherein a plurality of intermediate element parts each has an inner cone or surface and an outer cone or surface. In this connection in one embodiment it may be provided that the plurality of intermediate element parts each has an inner cone or surface and an outer cone or surface which are not formed coaxially with respect to one another. It is also possible to combine intermediate elements, wherein for one intermediate element the inner and outer cones or surfaces are formed coaxially and for a further intermediate element the inner and outer cones or surfaces are not formed coaxially. For example two intermediate element parts are provided which each have an inner cone or surface and an outer cone or surface. In both intermediate element parts the inner and outer cones or surfaces are not coaxial with respect to one another. The shape of the inner cone or surface of one intermediate element part is adapted to a receptacle of the outer cone of the other intermediate element part. The multi-part construction of the intermediate element increases the flexibility for the individual adaptation of the joint prosthesis system to the patient's situation. Alternatively, the intermediate element may be constructed in one piece. For individual adaptability to the patient, intermediate elements of different lengths can be supplied in to kit. It may also be provided that the longitudinal direction in a lower and an upper section of the intermediate element are at an angle relative to one another, so that for example the upper section is bent by comparison with the lower section.

In an advantageous embodiment of invention it may be provided that the outer shell is made of metal. The outer shell of the joint socket can be configured for cemented or non-cemented implantation.

An advantageous embodiment of the invention provides that the inlay is made of a plastic material. Polyethylene may be used for example as the plastic material.

A further development preferably provides that the inner cone or surface is formed on the intermediate element in a blind bore. The blind bore serves to receive the prosthesis neck in a mechanically secure manner.

In an advantageous embodiment of the invention it may be provided that one or more fastening elements is/are formed on the outer shell. Fastening pins which protect from the outer surface of the outer shell and/or screw plates may be provided for example as fastening elements. The screw plates serve to fasten the joint socket to the bone with the aid of one or more screws.

A modification of the invention may provide that the joint ball head is made of a ceramic material. Alternatively the joint ball head may be made of a metal material.

The explanations given in connection with advantageous embodiments of the joint prosthesis system apply correspondingly with regard to advantageous embodiments of the pre-assembled joint prosthesis system part. It may in particular be provided that the inlay is made of polyethylene. In one embodiment the intermediate element has one coupling element which is configured so as to be releasable coupled to a changing tool.

The pre-assembled joint prosthesis system part can also be formed with an intermediate element, wherein an outer cone or surface is releasably disposed in the associated inner cone or surface of the joint ball head and has an inner cone or surface to receive a further intermediate element or a prosthesis neck.

### DESCRIPTION OF PREFERRED EMBODIMENTS OF THE INVENTION

The invention is explained, below in greater detail on the basis of preferred embodiments with reference to the drawings, in the drawings:

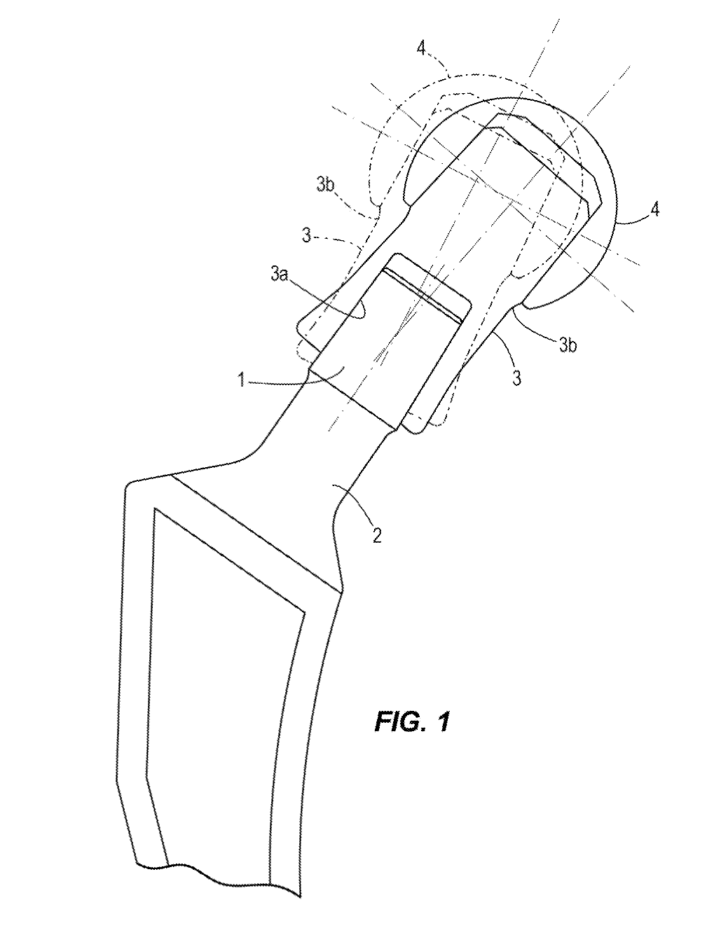

FIG. 1 shows a schematic representation of elements of a hip joint prosthesis system, i.e. a hip prosthesis, in which an intermediate element which is received in a joint ball head is fitted on a prosthesis neck,

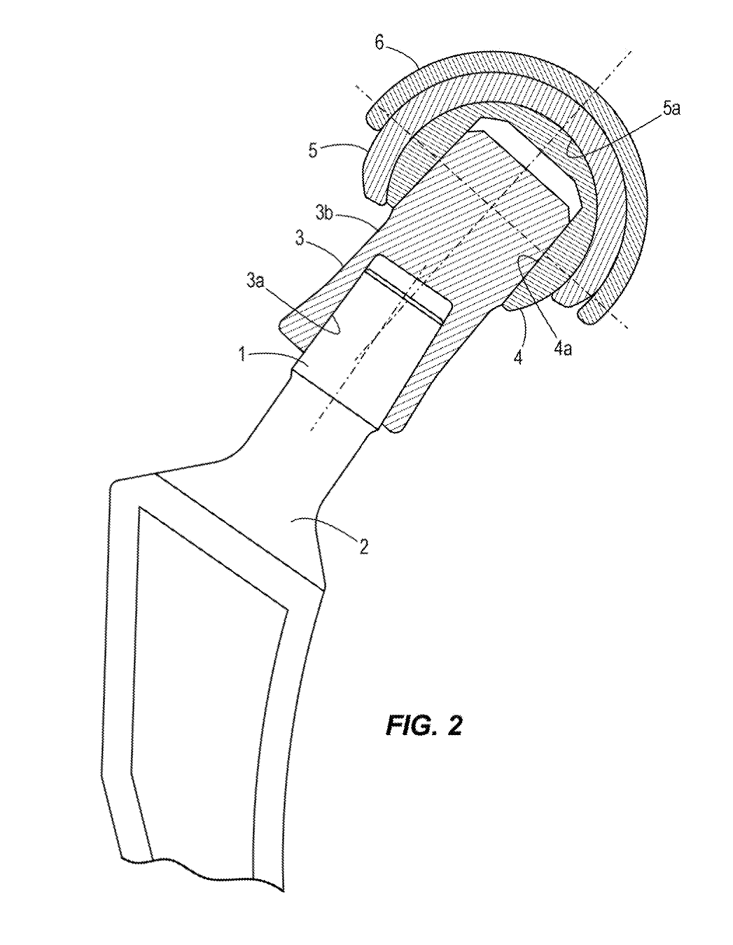

FIG. 2 shows a schematic representation of a hip joint prosthesis system in which the joint ball head is received in a bipolar joint socket,

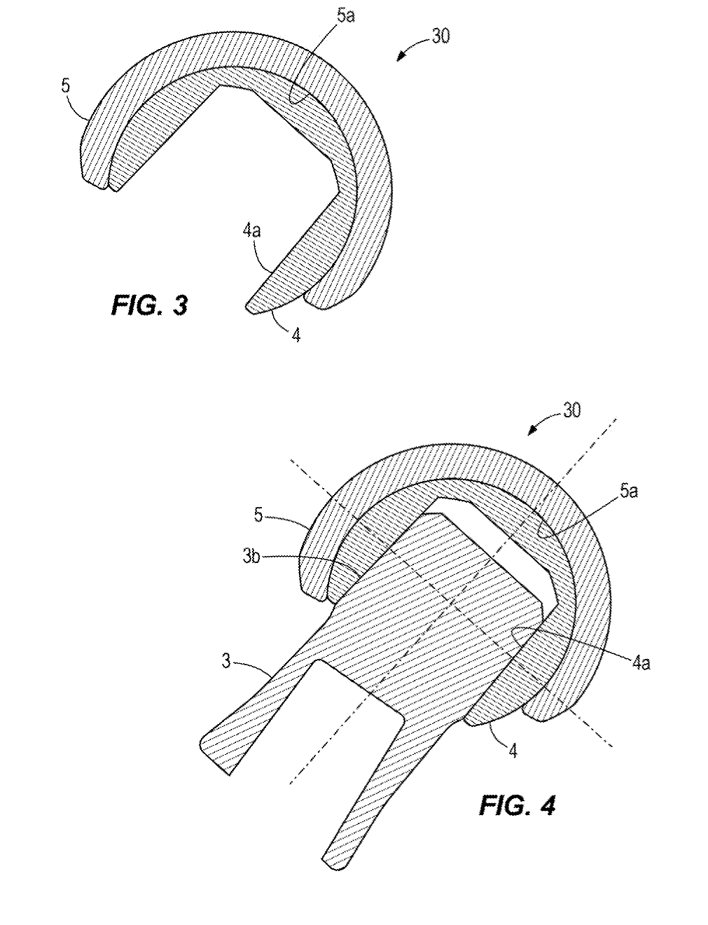

FIG. 3 shows a schematic representation of a pre-assembled joint prosthesis system part for a bipolar joint prosthesis system such as shown lot example in FIG. 2, and

FIG. 4 shows a schematic representation of a further pre-assembled joint prosthesis system part for a bipolar joint prosthesis system such as is shown for example in FIG. 2.

FIG. 1 shows a schematic representation of elements of a hip joint prosthesis system, in which an intermediate element 3 which is inserted into a joint ball head 4 is fitted onto a prosthesis neck 1 of a hip joint prosthesis 2. The representation shows the use of two different intermediate elements 3, which in different ways ensure that the center point of the joint ball head 4 is laterally offset with respect to the longitudinal direction of the prosthesis neck 2. Different types of embodiment of the intermediate element 3 which can be used in embodiments of the invention are disclosed per se for example in the documents DE 91 03 574 U1 and WO 2005/089676 A1. All the embodiments described there can be used in advantageous modifications of the present invention. Single-part or multi-part intermediate elements can be used. The intermediate elements each have an inner cone or surface 3a and an outer cone or surface 3b which may be formed coaxially or non-coaxially. FIG. 1 shows embodiments where the inner and outer cones or surfaces of the intermediate element are not formed coaxially.

FIG. 2 shows a schematic representation of a hip joint prosthesis system in which the joint ball head 4 is received movably in an inlay 5, wherein the inlay itself is disposed so as to be freely movable in an outer shell 6. The inlay 5 and the outer shell 6 form a bipolar joint socket. In this way an increased mobility of the hip joint prosthesis system is possible, since both the joint ball bead 4 within the inlay 5 and also the inlay 5 itself are movable in the outer shell 6. Because of the bipolar joint socket, the hip joint prosthesis system enables improved utilization of the options for adaptation which result from the use of the intermediate element 3.

FIGS. 3 and 4 shows as schematic representation of a pm-assembled joint prosthesis system part 30 for a bipolar joint prosthesis system such as is shown for example in FIG. 2. The joint prosthesis system part 30 thus pre-assembled may be assembled outside the operating area in a clean room environment and then placed in a sterile pack.

in the embodiment of the pre-assembled joint prosthesis system part 30 in FIG. 3 the joint ball head 4 is disposed as a plug head so as to be movable in the ball receptacle of the inlay 5 made of a plastic material, and so the joint ball bead is prepared to receive the prosthesis neck 2 which is still to take place during the operation, be it directly or indirectly by means of one or more intermediate elements 3.

In the embodiment of the pre-assembled joint prosthesis system part 30 in FIG. 4 the joint ball head 4 is disposed as a plug head so as to be movable m the ball receptacle 5a of the inlay 5 and in addition has an internal one or surface 4a in which the outer surface 3b of the intermediate element 3 is releasably received.

The features of the invention disclosed in the foregoing description, the claims and the drawings may be significant, both individually and in any combination, for the implementation of the invention in its different embodiments.

1. A modular joint prosthesis system, comprising:
an intermediate element which has an inner surface to receive a prosthesis neck and an outer surface,
a joint ball head which is formed as a plug head with an internal surface associated with and receiving the outer surface of the intermediate element, and
a bipolar joint socket in which an inlay is freely movably received in an outer shell and which has a ball receptacle associated with the joint ball head and configured to movably receive the joint ball head.

2. (canceled)

3. (canceled)

4. A joint prosthesis system as claimed in claim 1, wherein the outer shell is made of metal.

5. A joint prosthesis system as claimed in claim 1, wherein the inlay is made of a plastic material.

6. A joint prosthesis system as claimed in claim 1, wherein the inner surface is formed on the intermediate element in a blind bore.

7. A joint prosthesis system as claimed in claim 1, wherein the joint ball head is made of a metal material.

8. A joint prosthesis system as claimed in claim 1, wherein the joint ball head is made of a ceramic material.

9. A pre-assembled joint prosthesis system part for a modular joint prosthesis system, comprising an intermediate element which has an inner surface to receive a prosthesis neck and an outer surface, a joint ball head which is formed as a plug head with an internal surface associated with the outer surface of the intermediate element, and a bipolar joint socket, in which an inlay is freely movably received in an outer shell and which has a ball receptacle associated with the joint ball head and configured to movably receive the joint ball head; the pre-assembled joint prosthesis system part comprising:
an inlay made of a plastic material which is associated with a bipolar joint socket, is formed with a ball joint receptacle formed and is configured so as to be freely movably received in an outer shell of the bipolar joint socket, and
a ball joint head which is movably disposed as a plug head in the ball receptacle of the inlay and is formed with an inner surface.

10. A pre-assembled joint prosthesis system part as claimed in claim 9, comprising an intermediate element, wherein an outer surface of the intermediate element is releasably disposed in the associated inner surface of the joint ball head and the inner surface of the joint ball head receives one of an intermediate element and a prosthesis neck.

11. A pre-assembled joint prosthesis system part as claimed in claim 9, wherein the inlay made of polyethylene.

12. (canceled)

13. (canceled)

14. A sterile article having a pre-assembled joint prosthesis system part, comprising an inlay made of a plastic material which is associated with a bipolar joint socket, is formed with a ball receptacle formed and is configured so as to be freely movably received in an outer shell of the bipolar joint socket, and a joint ball head which is movably disposed as a plug head in the ball receptacle of the inlay and is formed with an inner surface, the sterile article comprising: an intermediate element, wherein an outer surface of the intermediate element is releasably disposed in the associated inner surface of the joint ball head and the inner surface of the joint ball head receives one of an intermediate element and a prosthesis neck, wherein the part is received in a sterile pack.
